(19) United States
(12) Patent Application Publication
(10) Pub. No.: US 2026/0201285 A1
(43) Pub. Date: Jul. 16, 2026

(54) MICROCAPSULES

(71) Applicant: GIVAUDAN SA, VERNIER (CH)

(72) Inventors: Vladica Bocokic, Argenteuil (FR); Marion DENIGOT, Argenteuil (FR); Ian Michael HARRISON, Argenteuil (FR)

(21) Appl. No.: 19/137,513

(22) PCT Filed: Dec. 15, 2023
(86) PCT No.: PCT/EP2023/086153

§ 371 Date: Jun. 10, 2025

(30) Foreign Application Priority Data
Dec. 20, 2022 (GB) 2219218.1

(51) Int. Cl. C11D 17/00; C11D 3/00; C11D 3/22; C11D 3/50

(52) U.S. Cl. CPC C11D 17/0039; C11D 3/001; C11D 3/226; C11D 3/505

(57) ABSTRACT

A core-shell microcapsule comprising
a) an inner shell encapsulating a benefit agent; and
b) an outer shell of crosslinked polysaccharide;
   crosslinking being effected by means of at least one oligofunctional (meth)acrylate compound.

The resulting core-shell microcapsules have a reduced content of material derived from non-renewable sources.

This disclosure relates to encapsulated compositions comprising at least one core-shell microcapsule. It also relates to a method for preparing such encapsulated compositions and to their use to enhance the performance of a benefit agent in a consumer product.

It is known to incorporate encapsulated benefit agents in consumer products, such as household care, personal care and fabric care products. Examples of benefit agents include fragrances, cosmetic agents, food ingredients, nutraceuticals, drugs and substrate enhancers.

Microcapsules that are particularly suitable for delivery of such benefit agents are core-shell microcapsules, wherein the core usually comprises the benefit agent and the shell is impervious or partially impervious to the benefit agent. Generally, these microcapsules are employed in aqueous media and the encapsulated benefit agents are hydrophobic. A broad selection of shell materials can be used, provided the shell material is impervious or partially impervious to the encapsulated benefit agent.

Benefit agents are encapsulated for a variety of reasons. Microcapsules can isolate and protect such materials from external suspending media, such as consumer product bases, in which they may be incompatible or unstable. They are also used to assist in the deposition of benefit agents onto substrates, such as skin or hair, or also fabrics or hard household surfaces in case of perfume ingredients. They can also act as a means of controlling the spatio-temporal release of a benefit agent.

A wide variety of encapsulating media as well as benefit agents suitable for the preparation of encapsulated compositions has been proposed in the prior art. Such encapsulating media include synthetic resins made from polyamides, polyureas, polyurethanes, polyacrylates, melamine-derived resins, or mixtures thereof. Aminoplast capsules, notably melamine-formaldehyde, have been found to be particularly good.

There is an increasing desire to provide microcapsules with a reduced proportion of materials obtained from non-renewable resources, such as synthetic petrochemicals. However, it has been found difficult to provide such capsules that can encapsulate with high encapsulation efficiency and that are sufficiently impervious to benefit agents during storage.

There is now provided a core-shell microcapsule comprising
(a) An inner shell encapsulating a benefit agent; and
(b) An outer shell of crosslinked polysaccharide;
crosslinking being effected by means of at least one oligofunctional (meth)acrylate compound.

By “benefit agent” is meant any substance which, when added to a product, may improve the perception of this product by a consumer or may enhance the action of this product in an application. Typical benefit agents include perfume ingredients, flavor ingredients, cosmetic ingredients, bioactive agents (such as bactericides, insect repellents and pheromones), substrate enhancers (such as silicones and brighteners), enzymes (such as lipases and proteases), dyes, pigments and nutraceuticals.

The inner shell of the microcapsule that encapsulates the benefit agent may be made of any suitable material. This will naturally depend on the nature of the benefit agent and the desired end-use. It may be a natural material, such as gelatin, or it may be one of the many synthetic materials known to and used by the art as a capsule wall former. Typical examples include (meth)acrylates, aminoplast resins, such as melamine- and urea-formaldehyde, and polyurea. A particular embodiment of inner shell is further detailed hereinunder.

The polysaccharide may be selected from any polysaccharide capable of crosslinking reactions with linker molecules that are oligofunctional (meth)acrylate compounds, further described hereinunder. Such polysaccharides are those comprising uronic acid units, that is, where a CH₂OH has been oxidized to form a COOH group. In a particular embodiment, the polysaccharide comprises hexuronic acid units. Polysaccharides having uronic acid units, in particular hexuronic acid units, are broadly available in nature.

The hexuronic acid units can be selected from the group consisting of galacturonic acid units, glucuronic acid units, in particular 4-O-methyl-glucuronic acid units, guluronic acid units and mannuronic acid units.

The polysaccharide comprising carboxylic acid groups may be branched. Branched polysaccharides comprising carboxylic acid groups have the advantage of forming more compact networks than linear polysaccharides and therefore may favor the imperviousness of the encapsulating shell, resulting in reduced leakage and greater encapsulation efficiency.

The carboxylic acid groups can be partially present in the form of the corresponding methyl ester. The percentage of carboxylic acid groups that are present in the form of the corresponding methyl ester can be from 3% to 95%, preferably from 4% to 75%, more preferably from 5 to 50%.

Alternatively, the percentage of carboxylic acid groups that are present in the form of the corresponding methyl ester can be less than 50%.

In the context of the present disclosure, polysaccharides comprising carboxylic acid groups, of which 50% or more are present in the form of the corresponding methyl ester, are referred to as “high methoxylated”. Polysaccharides comprising carboxylic acid groups, of which less than 50% are present in the form of the corresponding methyl ester, are referred to as “low methoxylated”.

The carboxylic acid groups can at least partially be present in the form of the corresponding carboxylate salt, in particular the corresponding sodium, potassium, magnesium or calcium carboxylate salt.

In an alternative embodiment of the disclosure, the carboxylic acid groups can at least partially be present in the form of a complex with a species selected from the group consisting of a zirconium species, a titanium species and a boron species, wherein the species are especially oxides.

Without being bound by any theory, it is surmised that presence of carboxylate salts or complexes in the polysaccharides limits their solubility in water and thereby promotes the formation of capsule shells. Furthermore, polyvalent metal species may promote intermolecular cross-linking, which may also improve the encapsulating properties of the shell.

The linker molecules are selected from oligofunctional acrylate or methacrylate compounds, and they may be selected from any such compound having a plurality of ethylenically-unsaturated terminal double bonds, typically from 2-6, particularly from 2-4 such bonds. While any such compound may be used, particular examples are the following:
3: 3-(Acryloyloxy)-2-hydroxypropyl methacrylate
15: ethylene glycol dimethacrylate
16: 1,3-Butylene Glycol Dimethacrylate
17: 1,3,5-triacryloylhexahydro-1,3,5-triazine
18: Tris(2-acryloyloxyethyl) Isocyanurate
19: pentaerythritol tetraacrylate

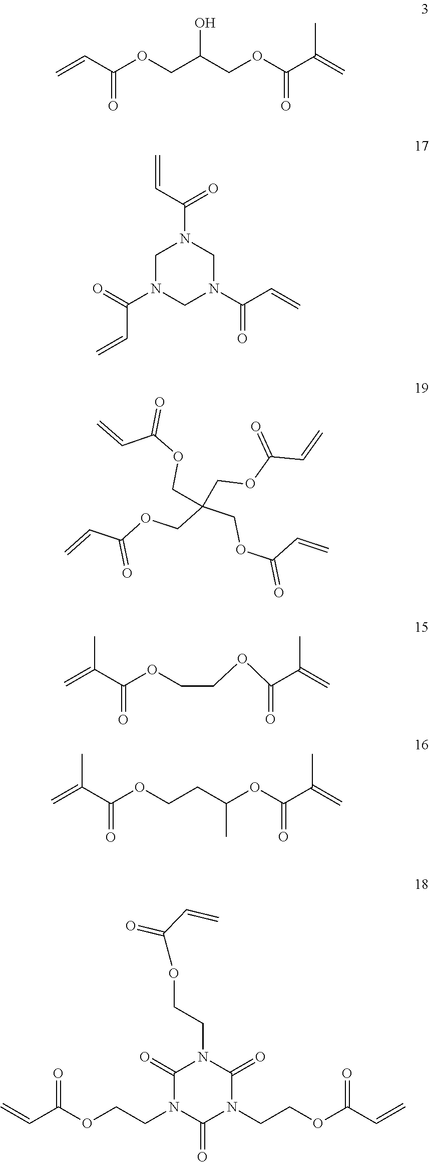

In a particular embodiment, the inner shell is formed from a reaction between an aminosilane and a polyfunctional diisocyanate.

The aminosilane may be selected from compounds of Formula (I).

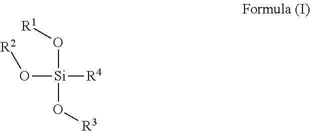

in which R¹, R² and R³ are each independently C₁-C₄ linear or branched alkyl or alkenyl residues, in particular methyl or ethyl, and R⁴ is a C₁-C₁₂, preferably a C₁-C₄, linear or branched alkyl or alkenyl residue comprising an amine functional group, in particular a primary, secondary or tertiary amine.

When the functional group is a primary amine, it can be a terminal primary amine. R⁴ is then preferably a C₁-C₅, even more preferably a C₁-C₄, linear terminal primary aminoalkyl residue. Specific aminosilanes of this category are selected from the group consisting of aminomethyltriethoxysilane, 2-aminoethyltriethoxysilane, 3-aminopropyltriethoxysilane, 4-aminobutyltri-ethoxysilane, 5-aminopentyltriethoxysilane, 6-aminohexyltriethoxysilane, 7-aminohptyltriethoxysilane and 8-aminooctyltriethoxysilane.

Without being bound by any theory, it is believed that the silane groups polycondense with one another to form a silica network at a liquid-liquid interface that additionally stabilizes this interface.

The aminosilane may be a bipodal aminosilane. By “bipodal aminosilane” is meant a molecule comprising at least one amino group and two residues, each of these residues bearing at least one alkoxysilane moiety.

In particular embodiments of the present disclosure, the at least one bipodal aminosilane has the Formula (II).

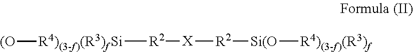

In the above Formula (II), X stands for —NR′—, —NR¹—CH₂—NR¹—, —NR¹—CH₂—CH₂—NR¹—, —NR′—CO—NR¹—, or

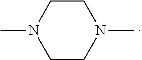

In the above Formula (II), R¹ each independently stand for H, CH₃ or C₂H₅. R² each independently stand for a linear or branched alkylene group with 1 to 6 carbon atoms. R³ each independently stand for a linear or branched alkyl group with 1 to 4 carbon atoms. R⁴ each independently stand for H or for a linear or branched alkyl group with 1 to 4 carbon atoms. f stands for 0, 1 or 2.

Bipodal aminosilanes are particularly advantageous for forming stable oil-water interfaces, compared to conventional silanes.

Examples of bipodal aminosilanes include, but are not limited to, bis(3-(triethoxysilyl)propyl)amine, N,N′-bis(3-(trimethoxysilyl)propyl)urea, bis(3-(methyldiethoxysilyl) propyl)amine, N,N′-bis(3-(trimethoxysilyl)propyl)ethane-1,2-diamine, bis(3-(methyldimethoxysilyl)propyl)-N-methylamine and N,N′-bis(3-(triethoxysilyl) propyl)piperazine.

The bipodal aminosilane can be a secondary aminosilane. Using a secondary bipodal aminosilane instead of primary aminosilane decreases the reactivity of the polymeric stabilizer with respect to electrophilic species, in particular aldehydes. Hence, benefit agents containing high levels of aldehydes may be encapsulated with a lower propensity for adverse interactions between core-forming and shell-forming materials.

In a particular embodiment, the secondary bipodal aminosilane is bis(3-(triethoxysilyl)propyl)amine. This particular secondary aminosilane has the advantage of releasing ethanol instead of more toxic and less desirable methanol during the polycondensation of the ethoxysilane groups.

Other aminosilanes may also be used in combination with the aforementioned bipodal aminosilanes, in particular the aminosilanes described hereinabove.

The aminosilane to polymeric surfactant weight ratio can be from 0.1 to 1.1, in particular from 0.2 to 0.9, even more particularly from 0.3 to 0.7, for example 0.5.

The polyfunctional isocyanate may be selected from alkyl, alicyclic, aromatic and alkylaromatic, as well as anionically modified polyfunctional isocyanates, with two or more (e.g. 3, 4, 5, etc.) isocyanate groups in a molecule.

Preferably, at least one polyfunctional isocyanate is an aromatic or an alkylaromatic polyfunctional isocyanate, the alkylaromatic polyfunctional isocyanate having preferably methylisocyanate groups attached to an aromatic ring. Both aromatic and methylisocyanate-substituted aromatic polyfunctional isocyanates have a superior reactivity compared to alkyl and alicyclic polyfunctional isocyanates. Among these, 2-ethylpropane-1,2,3-triyl tris((3-(isocyanatomethyl)phenyl)carbamate) is particularly preferred, because of its tripodal nature that favors the formation of intermolecular cross-links and because of its intermediate reactivity that favors network homogeneity. This alkylaromatic polyfunctional isocyanate is commercially available under the trademark Takenate™ D-100 N, sold by Mitsui or under the trademark Desmodur™ Quix175, sold by Covestro.

In a particular embodiment, the initial inner shell formation is stabilized in suspension in an aqueous phase by means of a polymeric stabilizer. Such a stabilizer is described in International Publication WO 2020/233887, but shall also be further described hereinunder.

The polymeric stabilizer is formed by a combination of a polymeric surfactant and at least one aminosilane. By “polymeric surfactant” is meant a polysaccharide or a mixture comprising at least one polysaccharide that has the property of lowering the interfacial tension between an oil phase and an aqueous phase, when dissolved in one or both of the phases.

In a particular embodiment of the disclosure, the polymeric stabilizer is formed by combination of pectin with bis(3-(triethoxysilyl)propyl)amine. Preferably, the polymeric stabilizer is formed by combination of pectin with bis(3-(triethoxysilyl)propyl)amine and 2-ethylpropane-1,2,3-triyl tris((3-(isocyanatomethyl)phenyl)carbamate). These combinations of natural polymeric surfactant and bipodal secondary aminosilane provide particularly advantageous interface stability and release properties. The stabilized interface is sufficiently impervious to effectively encapsulate the at least one benefit agent comprised in the core. The polymeric stabilizer effectively forms a shell encapsulating the at least one perfume ingredient comprised in the core.

Core-shell microcapsules according to the present disclosure generally have a volume average size (d50) of 1 to 100 μm, preferably 5 to 50 μm, even more preferably 10 to 30 μm.

In another aspect, the present disclosure relates to an encapsulated composition, in particular a composition as described herein above. The encapsulated composition comprises at least one core-shell microcapsule. The at least one core-shell microcapsule comprises a core comprising at least one benefit agent and a shell surrounding the core. The shell comprises a polymeric stabilizer that is formed by combination of a polymeric surfactant with at least one aminosilane. The shell additionally comprises a polysaccharide, preferably a polysaccharide comprising beta (1→4) linked monosaccharide units, even more preferably a cellulose derivative, in particular selected form the group consisting of hydroxyethyl cellulose, hydroxypropylmethyl cellulose, cellulose acetate and carboxymethyl cellulose, preferably hydroxyethyl cellulose.

In order to avoid any doubt, the polymeric stabilizer referred to in the foregoing paragraph does not need to be a polysaccharide comprising carboxylic acid groups. In case the polymeric stabilizer referred to in the foregoing paragraph is a polysaccharide comprising carboxylic acid groups, polysaccharide additionally comprised in the shell is a further polysaccharide.

It has been found that the polymeric stabilizer is a relevant factor to the balance between microcapsule stability with respect to both perfume leakage during storage and perfume release under in-use conditions. In particular, the importance of providing additional stabilization of the oil-water interface has been recognized. The polymeric stabilizer thus provides a stable platform, which allows for the addition of additional shell materials and/or shell precursors to form novel encapsulated perfume compositions. More specifically, the addition of a polysaccharide, preferably a polysaccharide comprising beta (1→4) linked monosaccharide units, even more preferably a cellulose derivative, leads to highly sustainable microcapsules with an excellent release profile.

The polysaccharide may be deposited on the outer surface of the capsule shell formed by the polymeric stabilizer. This results in a multilayer shell having at least one layer of polymeric stabilizer and one layer of polysaccharide. It may improve the imperviousness of the encapsulating shell by increasing the amount of encapsulating material.

To avoid any ambiguity, the present disclosure is by no means restricted to a shell having sharply-defined discrete layers, although this is one possible embodiment. More specifically, the layers can also be gradual and undiscrete. On the other hand, and at the other extreme, the shell can even be essentially homogenous.

The polysaccharide may react with unreacted isocyanate groups and increase the density of the cross-linked shell. But the polysaccharide may also interact with the polymeric stabilizer by physical forces, physical interactions, such as hydrogen bonding, ionic interactions, hydrophobic interactions or electron transfer interactions.

The shell additionally comprising a polysaccharide can be further stabilized with a stabilizing agent. Preferably the stabilizing agent comprises at least two carboxylic acid groups. Even more preferably, the stabilizing agent is selected from the group consisting of citric acid, benzene-1,3,5-tricarboxylic acid, 2,5-furandicarboxylic acid, itaconic acid, poly(itaconic acid) and combinations thereof.

Yet another aspect of the present disclosure relates to a method for preparing an encapsulated composition, in particular an encapsulated composition as described herein above. This method comprises the steps of:
a) Providing a polymeric surfactant;
b) Providing an aqueous phase;
c) Dissolving or dispersing the polymeric surfactant in the aqueous phase;
d) Providing at least one aminosilane;
e) Providing an oil phase comprising at least one benefit agent;
f) Optionally: Dissolving the at least one aminosilane in the oil phase;
g) Emulsifying the oil phase and the aqueous phase in presence of both of the polymeric surfactant and the aminosilane to form an emulsion of oil droplets in the aqueous phase;
h) Causing the at least one aminosilane and the polymeric surfactant to form a shell at the oil-water interface of the emulsified oil droplets, thereby forming a slurry of microcapsules;
i) Adding a polysaccharide, preferably a polysaccharide comprising beta (1→4) linked monosaccharide units, even more preferably a cellulose derivative, in particular selected form the group consisting of hydroxyethyl cellulose, hydroxpropylmethyl cellulose, cellulose acetate and carboxymethyl cellulose, preferably hydroxyethyl cellulose, to the microcapsule slurry formed in step h).
j) Cross-linking the polysaccharide with an oligo-acrylate/methacrylate

Oil-in-water emulsions have the advantage of providing a plurality of droplets that may be used as template for shell formation, wherein the shell is built around each of these droplets. Additionally, the droplet size distribution may be controlled in emulsions, by controlling the conditions of emulsifications, such as stirring speed and stirrer geometry. As a result, a plurality of microcapsules is obtained with controlled average size and size distribution, wherein the oil phase is encapsulated and forms thereby the core of the microcapsules.

In a variation of step i), instead of adding all of the polysaccharide at this step, some polysaccharide may be added at step g), it thus becoming incorporated into the shell formed by the aminosilane/polymeric surfactant shell. The oligo-acrylate/methacrylate then reacts with both the polysaccharide in the shell and the polysaccharide added at step i).

With respect to step h), the formation of the polymeric stabilizer is preferably initiated by adjusting the pH to a range of from 4.0 to 7.5, depending on the polymeric surfactant. For high methoxylated pectin, the optimal pH range is 6.5±0.5, for an alginate, the optimal pH range is 7.0±0.5 and for low methoxylated pectin and gum arabic, the optimal pH range is 4.5±0.5.

The temperature is preferably maintained at room temperature for at least 1 h, and then increased to at least 60° C., preferably at least 70° C., more preferably at least 80° C., but not more than 90° C., for example 85° C. Under these conditions, the formation of the shell is well controlled, meaning optimal stabilization of the interface is obtained.

The appropriate stirring speed and geometry of the mixer can be selected in order to obtain the desired average droplet size and droplet size distribution. It is a characteristic of the present disclosure that the polymeric stabilizer has sufficient interfacial activity and is able to promote the formation of dispersed oil droplets with desirable droplet size.

In a process according to the present disclosure, a one-liter vessel equipped with a turbine, or a cross-beam stirrer with pitched beam, such as a Mig stirrer, and having a stirrer diameter to reactor diameter of 0.6 to 0.8 may be used. Microcapsules can be formed in such reactor having a volume average size (d50) of 30 microns or less, more particularly 20 microns or less, at a stirring speed from about 100 to about 1200 rpm, more particularly from about 600 to 1000 rpm. Preferably, a Mig stirrer is used operating at a speed of 850+/−50 rpm. The person skilled in the art will however easily understand that such stirring conditions may change depending on the size of the reactor and of the batch size, on the exact geometry of the stirrer on the ratio of the diameter of the stirrer to the diameter of the reactor diameter ratios. For example, for a Mig stirrer with stirrer to reactor diameter ratio from 0.5 to 0.9 and slurry volumes ranging from 0.5 to 8 tons, the preferable agitation speed in the context of the present disclosure is from 150 rpm to 50 rpm.

In a particular embodiment of the present disclosure, the aminosilane to polymeric surfactant weight ratio in the emulsion is set within a range of from 0.1 to 1.1, more particularly from 0.2 to 0.9, still more particularly from 0.3 to 0.7, for example 0.35 or 0.65.

In a particular embodiment of the present disclosure, the shell material to oil weight ratio in the emulsion is set within a range from 0.01 to 0.5, more particularly from 0.025 to 0.4, even more particularly from 0.05 to 0.3.

Encapsulated compositions obtainable by the process mentioned hereinabove may be used as such or a polysaccharide, preferably a polysaccharide comprising beta (1→4) linked monosaccharide units, even more preferably a cellulose derivative, in particular selected form the group consisting of hydroxyethyl cellulose, hydroxpropylmethyl cellulose, cellulose acetate and carboxymethyl cellulose, preferably hydroxyethyl cellulose, may be added to the microcapsule shells formed in step h), as described in the above optional step i).

After formation of the microcapsules, the encapsulated composition is usually cooled to room temperature. Before, during or after cooling, the encapsulated composition may be further processed. Further processing may include treatment of the composition with anti-microbial preservatives, which preservatives are well known in the art. Further processing may also include the addition of a suspending aid, such as a hydrocolloid suspending aid to assist in the stable physical dispersion of the microcapsules and prevent any creaming or coalescence. Any additional adjuvants conventional in the art may also be added during further-processing.

In accordance with the process of the present disclosure, if desired, core-shell microcapsules may be further coated with a functional coating. A functional coating may entirely or only partially coat the microcapsule shell. Whether the functional coating is charged or uncharged, its primary purpose is to alter the surface properties of the microcapsule to achieve a desirable effect, such as to enhance the deposition of the microcapsule on a treated surface, such as a fabric, human skin or hair. Functional coatings may be post-coated to already formed microcapsules, or they may be physically incorporated into the microcapsule shell during shell formation. They may be attached to the shell by physical forces, physical interactions, such as hydrogen bonding, ionic interactions, hydrophobic interactions, electron transfer interactions, or they may be covalently bonded to the shell.

The at least one benefit agent can be at least one perfume ingredient. The at least one perfume ingredient can be selected from the group consisting of ADOXAL™ (2,6,10-trimethylundec-9-enal); AGRUMEX™ (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE ISO C 11 ((E)-undec-9-enal); ALDEHYDE MANDARINE 10%/TEC ((E)-dodec-2-enal); ALLYL AMYL GLYCOLATE (allyl 2-(isopentyloxy)acetate); ALLYL CYCLOHEXYL PROPIONATE (allyl 3-cyclohexylpropanoate); ALLYL OENANTHATE (allyl heptanoate); AMBER CORE™ (1-((2-(tert-butyl)cyclohexyl)oxy)butan-2-ol); AMBERMAX™ (1,3,4,5,6,7-hexahydro-beta,1,1,5,5-pentamethyl-2H-2,4a-methanonaphthal-ene-8-ethanol); AMYL SALICYLATE (pentyl 2-hydroxybenzoate); APHERMATE (1-(3,3-dimethylcyclohexyl)ethyl formate); BELAMBRE™ ((1R,2S,4R)-2′-isopropyl-1,7,7-trimethylspiro[bicyclo[2.2.1]heptane-2,4′-[1,3]dioxane]); BIGARYL (8-(sec-butyl)-5,6,7,8-tetrahydroquinoline); BOISAMBRENE FORTE™ ((ethoxymethoxy)cyclododecane); BOISIRIS™ ((1S,2R,5R)-2-ethoxy-2,6,6-trimethyl-9-methylenebicyclo[3.3.1]nonane); BORNYL ACETATE ((2S,4S)-1,7,7-trimethylbicyclo[2.2.1]heptan-2-yl acetate); BUTYL BUTYRO LACTATE (1-butoxy-1-oxopropan-2-yl butyrate); BUTYL CYCLOHEXYL ACETATE PARA (4-(tert-butyl)cyclohexyl acetate); CARYOPHYLLENE ((Z)-4,11,11-trimethyl-8-methylenebicyclo[7.2.0]undec-4-ene); CASHMERAN™ (1,1,2,3,3-pentamethyl-2,3,6,7-tetrahydro-1H-inden-4(5H)-one); CASSYRANE™ (5-tert-butyl-2-methyl-5-propyl-2H-furan); CITRAL ((E)-3,7-dimethylocta-2,6-dienal); CITRAL LEMAROME™ N ((E)-3,7-dimethylocta-2,6-dienal); CITRATHAL™ R((Z)-1,1-diethoxy-3,7-dimethylocta-2,6-diene); CITRONELLAL (3,7-dimethyloct-6-enal); CITRONELLOL (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CITRONELLYL PROPIONATE (3,7-dimethyloct-6-en-1-yl propionate); CLONAL (dodecanenitrile); CORANOL (4-cyclohexyl-2-methylbutan-2-ol); COSMONE™ ((Z)-3-methylcyclotetradec-5-enone); CYCLAMEN ALDEHYDE (3-(4-isopropylphenyl)-2-methylpropanal); CYCLOGALBANATE (allyl 2-(cyclohexyloxy)acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DAMASCONE DELTA ((E)-1-(2,6,6-trimethylcyclohex-3-en-1-yl)but-2-en-1-one); DECENAL-4-TRANS ((E)-dec-4-enal); DELPHONE (2-pentylcyclopentanone); DIHYDRO ANETHOLE (propanedioic acid 1-(1-(3,3-dimethylcyclohexyl)ethyl) 3-ethyl ester); DIHYDRO JASMONE (3-methyl-2-pentylcyclopent-2-enone); DIMETHYL BENZYL CARBINOL (2-methyl-1-phenylpropan-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butyrate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DUPICAL™ ((E)-4-((3aS,7aS)-hexahydro-1H-4,7-methanoinden-5(6H)-ylidene)butanal); EBANOL™ ((E)-3-methyl-5-(2,2,3-trimethylcyclopent-3-en-1-yl)pent-4-en-2-ol); ETHYL CAPROATE (ethyl hexanoate); ETHYL CAPRYLATE (ethyl octanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL OENANTHATE (ethyl heptanoate); ETHYL SAFRANATE (ethyl 2,6,6-trimethylcyclohexa-1,3-diene-1-carboxylate); EUCALYPTOL ((1s,4s)-1,3,3-trimethyl-2-oxabicyclo[2.2.2]octane); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FIXOLIDE™ (1-(3,5,5,6,8,8-hexamethyl-5,6,7,8-tetrahydronaphthalen-2-yl)ethanone); FLORALOZONE™ (3-(4-ethylphenyl)-2,2-dimethylpropanal); FLORHYDRAL (3-(3-isopropylphenyl)butanal); FLOROCYCLENE™ ((3aR,6S,7aS)-3a,4,5,6,7,7a-hexahydro-1H-4,7-methanoinden-6-yl propionate); FLOROPAL™ (2,4,6-trimethyl-4-phenyl-1,3-dioxane); FRESKOMENTHE™ (2-(sec-butyl)cyclohexanone); FRUITATE ((3aS,4S,7R,7aS)-ethyl octahydro-1H-4,7-methanoindene-3a-carboxylate); FRUTONILE (2-methyldecanenitrile); GALBANONE™ PURE (1-(3,3-dimethylcyclohex-1-en-1-yl)pent-4-en-1-one); GARDOCYCLENE™ ((3aR,6S,7aS)-3a,4,5,6,7,7a-hexahydro-1H-4,7-methanoinden-6-yl isobutyrate); GERANIOL ((E)-3,7-dimethylocta-2,6-dien-1-ol); GERANYL ACETATE SYNTHETIC ((E)-3,7-dimethylocta-2,6-dien-1-yl acetate); GERANYL ISOBUTYRATE ((E)-3,7-dimethylocta-2,6-dien-1-yl isobutyrate); GIVESCONE™ (ethyl 2-ethyl-6,6-dimethylcyclohex-2-enecarboxylate); HABANOLIDE™ ((E)-oxacyclohexadec-12-en-2-one); HEDIONE™ (methyl 3-oxo-2-pentylcyclopentaneacetate); HERBANATE™ ((2S)-ethyl 3-isopropylbicyclo[2.2.1]hept-5-ene-2-carboxylate); HEXENYL-3-CIS BUTYRATE ((Z)-hex-3-en-1-yl butyrate); HEXYL CINNAMIC ALDEHYDE ((E)-2-benzylideneoctanal); HEXYL ISOBUTYRATE (hexyl isobutyrate); HEXYL SALICYLATE (hexyl 2-hydroxybenzoate); INDOFLOR™ (4,4a,5,9b-tetrahydroindeno[1,2-d][1,3]dioxine); IONONE BETA ((E)-4-(2,6,6-trimethylcyclohex-1-en-1-yl)but-3-en-2-one); IRISONE ALPHA ((E)-4-(2,6,6-trimethylcyclohex-2-en-1-yl)but-3-en-2-one); IRONE ALPHA ((E)-4-(2,5,6,6-tetramethylcyclohex-2-en-1-yl)but-3-en-2-one); ISO E SUPER™ (1-(2,3,8,8-tetramethyl-1,2,3,4,5,6,7,8-octahydronaphthalen-2-yl)ethanone); ISOCYCLOCITRAL (2,4,6-trimethylcyclohex-3-enecarbaldehyde); ISONONYL ACETATE (3,5,5-trimethylhexyl acetate); ISOPROPYL METHYL-2-BUTYRATE (isopropyl 2-methyl butanoate); ISORALDEINE™ 70 ((E)-3-methyl-4-(2,6,6-trimethylcyclohex-2-en-1-yl)but-3-en-2-one); JASMACYCLENE™ ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); KARANAL™ (5-(sec-butyl)-2-(2,4-dimethylcyclohex-3-en-1-yl)-5-methyl-1,3-dioxane); KOAVONE ((Z)-3,4,5,6,6-pentamethylhept-3-en-2-one); LEAF ACETAL ((Z)-1-(1-ethoxyethoxy)hex-3-ene); LEMONILE™ ((2E,6Z)-3,7-dimethylnona-2,6-dienenitrile); LIFFAROME™ GIV ((Z)-hex-3-en-1-yl methyl carbonate); LILIAL™ (3-(4-(tert-butyl)phenyl)-2-methylpropanal); LINALOOL (3,7-dimethylocta-1,6-dien-3-ol); LINALYL ACETATE (3,7-dimethylocta-1,6-dien-3-yl acetate); MAHONIAL™ ((4E)-9-hydroxy-5,9-dimethyl-4-decenal); MALTYL ISOBUTYRATE (2-methyl-4-oxo-4H-pyran-3-yl isobutyrate); MANZANATE (ethyl 2-methylpentanoate); MELONAL™ (2,6-dimethylhept-5-enal); MENTHOL (2-isopropyl-5-methylcyclohexanol); MENTHONE (2-isopropyl-5-methylcyclohexanone); METHYL CEDRYL KETONE (1-((1S,8aS)-1,4,4,6-tetramethyl-2,3,3a,4,5,8-hexahydro-1H-5,8a-methanoazulen-7-yl)ethanone); METHYL NONYL KETONE EXTRA (undecan-2-one); METHYL OCTYNE CARBONATE (methyl non-2-ynoate); METHYL PAMPLEMOUSSE (6,6-dimethoxy-2,5,5-trimethylhex-2-ene); MYRALDENE (4-(4-methylpent-3-en-1-yl)cyclohex-3-enecarbaldehyde); NECTARYL (2-(2-(4-methylcyclohex-3-en-1-yl)propyl)cyclopentanone); NEOBERGAMATE™ FORTE (2-methyl-6-methyleneoct-7-en-2-yl acetate); NEOFOLIONE™ ((E)-methyl non-2-enoate); NEROLIDYLE™ ((Z)-3,7,11-trimethyldodeca-1,6,10-trien-3-yl acetate); NERYL ACETATE HC ((Z)-3,7-dimethylocta-2,6-dien-1-yl acetate); NONADYL (6,8-dimethylnonan-2-ol); NONENAL-6-CIS ((Z)-non-6-enal); NYMPHEAL™ (3-(4-isobutyl-2-methylphenyl)propanal); ORIVONE™ (4-(tert-pentyl)cyclohexanone); PARADISAMIDE™ (2-ethyl-N-methyl-N-(m-tolyl)butanamide); PELARGENE (2-methyl-4-methylene-6-phenyltetrahydro-2H-pyran); PEONILE™ (2-cyclohexylidene-2-phenylacetonitrile); PETALIA™ (2-cyclohexylidene-2-(o-tolyl)acetonitrile); PIVAROSE™ (2,2-dimethyl-2-pheylethyl propanoate); PRECYCLEMONE™ B (1-methyl-4-(4-methylpent-3-en-1-yl)cyclohex-3-enecarbaldehyde); PYRALONE™ (6-(sec-butyl)quinoline); RADJANOL™ SUPER ((E)-2-ethyl-4-(2,2,3-trimethylcyclopent-3-en-1-yl)but-2-en-1-ol); RASPBERRY KETONE (N112) (4-(4-hydroxyphenyl)butan-2-one); RHUBAFURANE™ (2,2,5-trimethyl-5-pentylcyclopentanone); ROSACETOL (2,2,2-trichloro-1-phenylethyl acetate); ROSALVA (dec-9-en-1-ol); ROSYFOLIA ((1-methyl-2-(5-methylhex-4-en-2-yl)cyclopropyl)-methanol); ROSYRANE™ SUPER (4-methylene-2-phenyltetrahydro-2H-pyran); SERENOLIDE (2-(1-(3,3-dimethylcyclohexyl)ethoxy)-2-methylpropyl cyclopropanecarboxylate); SILVIAL™ (3(4 isobutylphenyl)-2-methylpropanal); SPIROGALBANONE™ (1-(spiro[4.5]dec-6-en-7-yl)pent-4-en-1-one); STEMONE™ ((E)-5-methylheptan-3-one oxime); SUPER MUGUET™ ((E)-6-ethyl-3-methyloct-6-en-1-ol); SYLKOLIDE™ ((E)-2-((3,5-dimethylhex-3-en-2-yl)oxy)-2-methylpropyl cyclopropanecarboxylate); TERPINENE GAMMA (1-methyl-4-propan-2-ylcyclohexa-1,4-diene); TERPINOLENE (1-methyl-4-(propan-2-ylidene)cyclohex-1-ene); TERPINYL ACETATE (2-(4-methylcyclohex-3-en-1-yl)propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); TRIDECENE-2-NITRILE ((E)-tridec-2-enenitrile); UNDECAVERTOL ((E)-4-methyldec-3-en-5-ol); VELOUTONE™ (2,2,5-trimethyl-5-pentylcyclopentanone); VIRIDINE™ ((22 dimethoxyethyl)benzene); ZINARINE™ (2-(2,4-dimethylcyclohexyl)pyridine); and mixtures thereof.

A comprehensive list of perfume ingredients that may be encapsulated in accordance with the present disclosure can be found in the perfumery literature, for example “Perfume & Flavor Chemicals”, S. Arctander, Allured Publishing, 2000.

The at least one benefit agent can also be a cosmetic ingredient. Preferably, the cosmetic ingredients have a calculated octanol/water partition coefficient (C log P) of 1.5 or more, more preferably 3 or more. Alternatively preferred, the C log P of the cosmetic ingredient is from 2 to 7.

Particularly useful cosmetic ingredients may be selected from the group consisting of emollients, smoothening actives, hydrating actives, soothing and relaxing actives, decorative actives, anti-aging actives, draining actives, remodelling actives, skin levelling actives, preservatives, anti-oxidant actives, antibacterial or bacteriostatic actives, cleansing actives, lubricating actives, structuring actives, hair conditioning actives, whitening actives, texturing actives, softening actives, anti-dandruff actives and exfoliating actives.

Particularly useful cosmetic ingredients include, but are not limited to, hydrophobic polymers, such as alkyldimethylsiloxanes, polymethyl silsesquioxanes, polyethylene, polyisobutylene, styrene-ethylene-styrene and styrene-butylene-styrene block copolymers, mineral oils, such as hydrogenated isoparaffins, silicone oils, vegetable oils, such as argan oil, jojoba oil, aloe vera oil, fatty acids and fatty alcohols and their esters, glycolipides, phospholipides, sphingolipides, such as ceramides, sterols and steroids, terpenes, sesquiterpenes, triterpenes and their derivatives, essential oils, such as arnica oil, artemisia oil, bark tree oil, birch leaf oil, calendula oil, cinnamon oil, echinacea oil, eucalyptus oil, ginseng oil, jujube oil, helianthus oil, jasmine oil, lavender oil, lotus seed oil, perilla oil, rosmary oil, sandal wood oil, tea tree oil, thyme oil, valerian oil, wormwood oil, ylang ylang oil and yucca oil.

The resultant encapsulated composition, presented in the form of a slurry of microcapsules suspended in an aqueous suspending medium may be incorporated as such in a consumer product base. If desired, however, the slurry may be dried to present the encapsulated composition in dry powder form. Drying of a slurry of microcapsules is conventional, and may be carried out according techniques known in the art, such as spray-drying, evaporation, lyophilization or use of a desiccant. Typically, as is conventional in the art, dried microcapsules will be dispersed or suspended in a suitable powder, such as powdered silica, which can act as a bulking agent or flow aid. Such suitable powder may be added to the encapsulated composition before, during or after the drying step.

A further aspect of the disclosure relates to an encapsulated composition obtainable any of the methods described herein above.

Yet another aspect of the disclosure relates to a use of an encapsulated composition as described herein above to enhance the performance of a benefit agent in a consumer product.

The disclosure also relates to a consumer product comprising an encapsulated composition as described hereinabove. The consumer product may be selected from the group consisting of fabric care detergents and conditioners, hair care conditioners, shampoos, heavy duty liquid detergents, hard surface cleaners, detergent powders, soaps, shower gels and skin care products, particularly fabric softeners and hair conditioners.

Encapsulated compositions according to the present disclosure are particularly useful when employed as perfume delivery vehicles in consumer goods that require, for delivering optimal perfumery benefits, that the microcapsules adhere well to a substrate on which they are applied. Such consumer goods include hair shampoos and conditioners, as well as textile-treatment products, such as laundry detergents and conditioners.

A further aspect of the present disclosure relates to a polymeric stabilizer formed by combination of a polymeric surfactant with at least one aminosilane, in particular an aminosilane as described herein above. The polymeric surfactant comprises a polysaccharide comprising carboxylic acid groups and is in particular a polymeric surfactant as described herein above.

Yet another aspect of the present disclosure relates to a use of a polymeric stabilizer as described herein above in the encapsulation of a benefit agent. The polymeric stabilizer stabilizes the oil/water interfaces and, thereby, provides a template for the preparation of encapsulated perfume and/or cosmetic compositions.

The present disclosure also relates to a method for enhancing the performance of a benefit agent in a consumer product by adding an encapsulated composition according to the present disclosure.

Furthermore, the present disclosure refers to a method of encapsulating a benefit agent, wherein the polymeric stabilizer as described herein above stabilizes and encapsulates the oil droplets of the oil in water emulsion, and wherein the oil phase comprises the at least one benefit agent.

The disclosure is further described by the following examples, which describe particular embodiments, and which are not to be regarded as being in any way limiting.

### EXAMPLE 1

### General Preparation

In a 100 g reactor equipped with a mechanical overhead stirrer, 11.5 g of perfume (part 1) were mixed with 0.7 g of bis(3-(triethoxysilyl)propyl)amine (CAS 13497-18-2) and 0.5 g of Takenate™ D110N (1,3-propanediol, 2-ethyl-2-(hydroxymethyl)-, polymer with bis(isocyanatomethyl)-cyclohexatriene (CAS 51852-81-4)) under mild stirring at ambient temperature. 27 g of fragrance was further added to this mixture under stirring, and 66 g of water then carefully added with the stirring stopped. The stirring was restarted to create an emulsion, to which 1.35 g of pectin (Roeper APA 104 high methoxy) was added as a powder. The temperature was then increased to 45° C. for 30 minutes and maintained for 2 h, followed by a further increase in temperature to 55° C., where it was maintained for 1 h. The heating was increased again towards 85° C., and at 70° C., 0.3 g of trimesic acid were added as powder. At 85° C., 2.3 g of starch (HiCap™ 100), 0.4 g of linker acrylate (selected from those shown below) and 60 μl of radical initiator (selected from those shown below) as 4 mass % aqueous solution were added. 30 minutes later another 60 μl of radical initiator solution was added, and the temperature increased to 90° C., where it was maintained for 1 h 30.

The heating was stopped and reaction mixture was allowed to cool down gradually to room temperature.

The initiators used were the following:

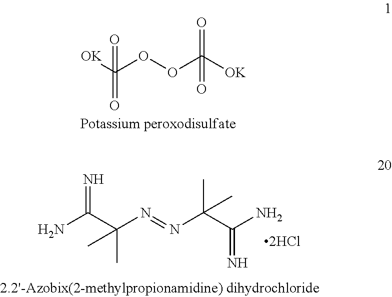

The linker molecules were the following:

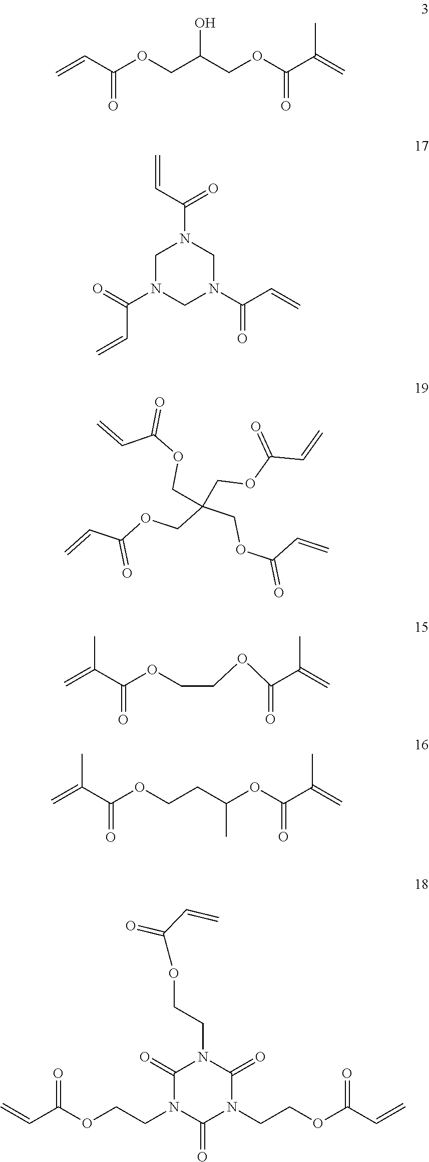

The specific combinations used in the general preparation were as follows:

| | | Nr. reactive | | |
|---|---|---|---|---|
| Nr. | Linker | sites | Initiator | Polysaccharide |
| 1 | 3 | 2 | 1 | HiCap100 |
| 2 | 19 | 4 | 1 | HiCap100 |
| 3 | 17 | 3 | 1 | HiCap100 |
| 4 | 15 | 2 | 1 | HiCap100 |
| 5 | 3 | 2 | 1 | — |
| 6 | 3 | 2 | 20 | HiCap100 |
| 7 | 15 | 2 | 20 | HiCap100 |
| 8 | 18 | 3 | 1 | HiCap100 |
| 9 | — | — | — | HiCap100 |
| 10 | 16 | 2 | 1 | HiCap100 |

The results of these preparations were slurries of fragrance-containing microcapsules in the size range of 5-50 μm.

The typical characterisations performed afterwards included the size distribution measurement by light diffraction, solid content residue upon drying at 120° C., perfume extraction upon maceration of capsules dispersed in a liquid softener base at 37° C., and the determination of the polymer residue in the liquid phase by size exclusion chromatography.

### Evaluation of Fragrance Stability in Fabric Softener

Capsules as prepared above were tested for stability in an unperfumed liquid fabric softener base (“LFS”). Each of the capsule samples was added to a sample of the LFS at 0.5 to 1.0% of the capsule slurry by weight and incorporated by stirring at room temperature for 10 minutes. The samples were assessed visually with respect to their mechanical integrity and fragrance content using a microscope at 4× and 10× magnification immediately after the incorporation into the base (t0), and also after being macerated at a temperature of 37° C. for 5 days (t5). The changes between t0 and t5, of the capsule shape, and particularly of the fragrance content in the capsule, were compared in order to obtain their stability. The amount of the still-encapsulated fragrance in the capsules was determined visually by image analysis. The visual determination of the still encapsulated fragrance is within +5% of the value determined by the gas chromatography (GC-MS) methods (after suitable extraction of the fragrance from the sample.

The results are shown in the following table.

| | | | Polymer incorporated in the |
|---|---|---|---|
| Ex. | Yield | Stability | shell (% of initial polymer |
| Nr | (%) | in LFS* | amount) |
| 1 | 100 | fail | n.d. |
| 2 | 100 | pass | 70 |
| 3 | 100 | pass | 35 |
| 4 | 100 | pass | 60 |
| 5 | 97 | fail | nd |
| 6 | 100 | pass | 75 |
| 7 | 100 | pass | 80 |
| 8 | 100 | fail | n.d. |
| 9 | 100 | pass | 30 |
| 10 | 100 | n.d. | n.d. |

*Liquid fabric softener, 5 days at 37° C.;
scale legend:
pass = more than 60% fragrance retained;
fail = more than 60% fragrance extracted in the base.
n.d. = not determined.

The proportion of polymer incorporated in the capsule shell (as a percentage of the total polymer used) is measured by the proportion of free (unreacted) polymer in the continuous medium.

1. A core-shell microcapsule comprising
a) an inner shell encapsulating a benefit agent; and
b) an outer shell of crosslinked polysaccharide;
crosslinking being effected by means of at least one oligofunctional (meth)acrylate compound.

2. A core-shell capsule according to claim 1, in which the polysaccharide comprises uronic acid units.

3. A core-shell capsule according to claim 2, in which the uronic acid units are hexuronic acid units, particularly hexuronic units selected from the group consisting of galacturonic acid units and glucuronic acid units, more particularly 4-O-methyl-glucuronic acid units, guluronic acid units and mannuronic acid units.

4. A core-shell capsule according to claim 1, in which the oligofunctional (meth)acrylate compound is selected from the group consisting of 3-(acryloyloxy)-2-hydroxypropyl methacrylate, ethylene glycol dimethacrylate, 1,3-butylene glycol dimethacrylate, 3,5-triacryloylhexahydro-1,3,5-triazine, tris(2-acryloyloxyethyl) isocyanurate and pentaerythritol tetraacrylate.

5. A core-shell capsule according to claim 1, in which the inner shell is formed from a reaction between a silane and a polyfunctional diisocyanate.

6. A core-shell capsule according to claim 1, in which the initial shell is stabilised in aqueous suspension by a polymeric stabilizer that is the product of a polymeric surfactant and an aminosilane, the polymeric surfactant comprising a polysaccharide or a mixture comprising at least one polysaccharide that has the property of lowering the interfacial tension between an oil phase and an aqueous phase, when dissolved in one or both of the phases.

7. A core-shell capsule according to claim 6, in which the polymeric stabilizer is formed by combination of pectin with bis(3-(triethoxysilyl)propyl)amine or a mixture of bis(3-(triethoxysilyl)propyl)amine and 2-ethylpropane-1,2,3-triyl tris((3-(isocyanatomethyl)phenyl)carbamate).

8. A method for preparing an encapsulated composition, in particular an encapsulated composition as described herein above. This method comprises the steps of:
(a) Providing a polymeric surfactant;
(b) Providing an aqueous phase;
(c) Dissolving or dispersing the polymeric surfactant in the aqueous phase;
(d) Providing at least one aminosilane;
(e) Providing an oil phase comprising at least one benefit agent;
(f) Optionally: Dissolving the at least one aminosilane in the oil phase;
(g) Emulsifying the oil phase and the aqueous phase in presence of both of the polymeric surfactant and the aminosilane to form an emulsion of oil droplets in the aqueous phase;
(h) Causing the at least one aminosilane and the polymeric surfactant to form a shell at the oil-water interface of the emulsified oil droplets, thereby forming a slurry of microcapsules;
(i) Adding a polysaccharide, preferably a polysaccharide comprising beta (1→4) linked monosaccharide units, even more preferably a cellulose derivative, in particular selected form the group consisting of hydroxyethyl cellulose, hydroxpropylmethyl cellulose, cellulose acetate and carboxymethyl cellulose, preferably hydroxyethyl cellulose, to the microcapsule slurry formed in step h).
(j) Cross-linking the polysaccharide with an oligo-acrylate/methacrylate
